(19) United States
(12) Patent Application Publication
(10) Pub. No.: US 2003/0055499 A1
(43) Pub. Date: Mar. 20, 2003

(54) Low profile intraocular lenses

(76) Inventors: Tuan Anh Nguyen, Orange, CA (US); Laurnt G. Hoffmann, Foothill Ranch, CA (US); Donald C. Stenger, Anaheim Hills, CA (US)

Correspondence Address: BAUSCH & LOMB, INC., ONE BAUSCH & LOMB PLACE, ROCHESTER, NY, 14604-2701, US

(21) Appl. No.: 09/952,855

(22) Filed: Sep. 14, 2001

(51) Int. Cl. A61F 2/16

(52) U.S. Cl. 623/6.49

(57) ABSTRACT

A refractive intraocular lens including an optic portion having an outer peripheral edge and two or more but preferably two, three or four equally spaced haptic elements. Each haptic element is of like fluidly varying trapezoidal form to achieve a “propeller-like” appearance. Each haptic element is also manufactured to have an inner portion with a gusset and an outer portion with a contact button for supporting the optic portion in a patient's eye. The inner portion of each haptic element is permanently connected to the outer peripheral edge of the optic portion. Each haptic is formed to have a relatively low profile and a greater resistance to bending in a plane generally parallel to an eye's optical axis than in a plane generally perpendicular to the eye's optical axis. The intraocular lens is so designed to exhibit less than approximately 1.0 mm axial displacement of the optic portion along the eye's optical axis under a compression force suitable to effect a 1.0 mm in diameter compression of the intraocular lens.

### FIELD OF THE INVENTION

The present invention relates to intraocular lenses (IOLs) and a method for making and using the same. More particularly, the present invention relates to IOLs designed primarily for refractive correction in phakic eyes where the eye's natural lens remains intact. IOLs made in accordance with the present invention may also be used in aphakic eyes where a diseased natural lens is surgically removed, such as in the case of cataracts.

### BACKGROUND OF THE INVENTION

Visual acuity deficiencies such as myopia (nearsightedness), hyperopia (farsightedness) and presbyopia (age-related farsightedness) are typically corrected through the use of refractive lenses such as spectacles or contact lenses. Although these types of lenses are effective in correcting a wearer's eyesight, many wearers consider the lenses inconvenient. The lenses must be located, worn at certain times, removed periodically and may be lost or misplaced. The lenses may also be dangerous or cumbersome if the wearer participates in athletic activities or suffers an impact in an area near the eyes.

The use of surgically implanted IOLs as a permanent form of refractive correction has been gaining in popularity. IOL implants have been used for years in aphakic eyes as replacements for diseased natural crystalline lenses that have been surgically removed from the eyes. Many different IOL designs have been developed over past years and proven successful for use in aphakic eyes. The successful IOL designs to date primarily include an optic portion with supports therefor, called haptics, connected to and surrounding at least part of the optic portion. The haptic portions of an IOL are designed to support the optic portion of the IOL in the lens capsule, anterior chamber or posterior chamber of an eye.

Commercially successful IOLs have been made from a variety of biocompatible materials, ranging from more rigid materials such as polymethylmethacrylate (PMMA) to softer, more flexible materials capable of being folded or compressed such as silicones and certain acrylics. Haptic portions of the IOLs have been formed separately from the optic portion and later connected thereto through processes such as heat, physical staking and/or chemical bonding. Haptics have also been formed as an integral part of the optic portion in what is commonly referred to as “single-piece” IOLs.

Softer, more flexible IOLs have gained in popularity in recent years due to their ability to be compressed, folded, rolled or otherwise deformed. Such softer IOLs may be deformed prior to insertion thereof through an incision in the cornea of an eye. Following insertion of the IOL in an eye, the IOL returns to its original pre-deformed shape due to the memory characteristics of the soft material.

Softer, more flexible IOLs as just described may be implanted into an eye through an incision that is much smaller, i.e., 2.8 to 3.2 mm, than that necessary for more rigid IOLs, i.e., 4.8 to 6.0 mm. A larger incision is necessary for more rigid IOLs because the lens must be inserted through an incision in the cornea slightly larger than that of the diameter of the inflexible IOL optic portion. Accordingly, more rigid IOLs have become less popular in the market since larger incisions have been found to be associated with an increased incidence of postoperative complications, such as induced astigmatism.

After IOL implantation, both softer and more rigid IOLs are subject to compressive forces exerted on the outer edges thereof, which typically occur when an individual squints or rubs the eye. These compressive forces may result in decentration of the IOL and distortion of the visual image. Compressive forces exerted on an IOL also tend to cause axial displacement of the IOL along the optical axis of an eye. Movement of an IOL along the optical axis of an eye has the potential to cause the IOL to contact and damage the delicate corneal endothelial cell layer of the eye. Also, IOLs of current designs, whether formed of either softer or more rigid materials, tend to deflect along the optical axis of an eye when the haptics are compressed. IOL manufacturers provide a wide range of IOL sizes to more precisely fit IOLs to each particular patient's eye size. Providing a wide range of IOL sizes is an attempt to minimize the potential for haptic compression and the associated axial displacement of the IOL optic along the optical axis of an eye.

Haptic element contact with delicate tissues of the anterior chamber of an eye and movement of the haptic elements within the eye are of particular concern with phakic IOLs. Haptic element contact and movement that causes tissue trauma within the angle of the anterior chamber has been known to induce glaucoma and potentially blindness.

Because of the noted shortcomings of past and current IOL designs, there is a need for phakic IOLs designed to minimize contact and movement within the angle of the anterior chamber. Also, there is a need for phakic IOLs designed to minimize axial displacement of the IOL optic portion along the optical axis of the eye when compressive forces are exerted against the outer edges thereof. By lessening an IOLs movement along the optical axis of an eye, more certain refractive correction may be achieved and the risk of endothelial cell layer damage may be reduced.

### SUMMARY OF THE INVENTION

An intraocular lens (IOL) made in accordance with the present invention has an optic portion with an outer peripheral edge and two or more but preferably two, three or four equally spaced haptic elements for supporting the optic portion in a patient's eye. Preferably, each of the haptic elements is of like form to achieve a “propeller” effect for ease of implantation, turning and centering of the IOL and to achieve the desired flexation with optic portion rotation as described in greater detail below. Each of the subject haptic elements is of relatively thin or “low profile” form and has an inner portion and an outer portion. The inner portion of each haptic element includes a gusset portion permanently connected to the outer peripheral edge of the optic portion. The outer portion of each haptic element includes a softened contact button. The softened contact button is a relatively small rounded protuberance to ensure minimal contact with a patient's eye tissue. Extending between the gusset portion and contact button, each haptic element includes an elongated arcuate central portion. From the gusset portion throughout the length of the elongated arcuate central portion, the dimensions of the haptic element vary to achieve a low profile and enable flexation with optic portion rotation. The haptic element flexation of the subject IOL provides greater resistance to bending in a plane generally parallel to the optical axis of an eye than in a plane generally perpendicular thereto. This flexibility characteristic of the haptic elements causes the optic portion to rotate in a release of any compression forces exerted against the haptic elements of the IOL. Through haptic element flexation and optic portion rotation, axial displacement of the optic portion along the optical axis of an eye is eliminated. With relatively thin or low profile haptic elements and the described flexibility characteristic, the present IOL provides enhanced fit within the anterior chamber of a phakic eye. Likewise, the relatively thin or low profile haptic elements provide enhanced safety within the eye by better avoiding contact with delicate eye tissues such as the corneal endothelium.

Accordingly, it is an object of the present invention to provide intraocular lenses for use in phakic eyes.

Another object of the present invention is to provide intraocular lenses for use in phakic eyes, which are relatively thin or have a relatively low profile.

Another object of the present invention is to provide intraocular lenses for use in phakic eyes, which minimize axial displacement of the optic portions of the lenses along the optical axis of the eyes.

Another object of the present invention is to provide intraocular lenses that allow for increased ease of implantation, turning and centering of the same.

Another object of the present invention is to provide intraocular lenses for use in phakic eyes, which minimize damage to tissues in the interior of the eyes.

Still another object of the present invention is to provide intraocular lenses, which are resistant to decentration within the eyes.

These and other objectives and advantages of the present invention, some of which are specifically described and others that are not, will become apparent from the detailed description, drawings and claims that follow, wherein like features are designated by like numerals.

### BRIEF DESCRIPTION OF THE DRAWINGS

FIG. 1 is a schematic representation of the interior of a human eye including a natural lens and a refractive IOL implanted in the anterior chamber of the eye;

FIG. 2 is a plan view of an IOL with three haptics made in accordance with the present invention;

FIG. 3 is a side view of the IOL of FIG. 2;

FIG. 4 is a cross sectional view of a haptic element of the IOL of FIG. 2 taken along line 4-4;

FIG. 5 is a cross sectional view of a haptic element of the IOL of FIG. 2 taken along line 5-5;

FIG. 6 is a cross sectional view of a haptic element of the IOL of FIG. 2 taken along line 6-6;

FIG. 7 is a cross sectional view of a haptic element of the IOL of FIG. 2 taken along line 7-7;

FIG. 8 is a cross sectional view of a haptic element of the IOL of FIG. 2 taken along line 8-8;

FIG. 9 is a plan view of an IOL with four haptics made in accordance with the present invention;

FIG. 10 is a plan view of an IOL with two haptics made in accordance with the present invention; and

FIG. 11 is a cross-sectional view of a haptic element of the IOL of FIG. 2 taken along line 11-11.

### DETAILED DESCRIPTION OF THE INVENTION

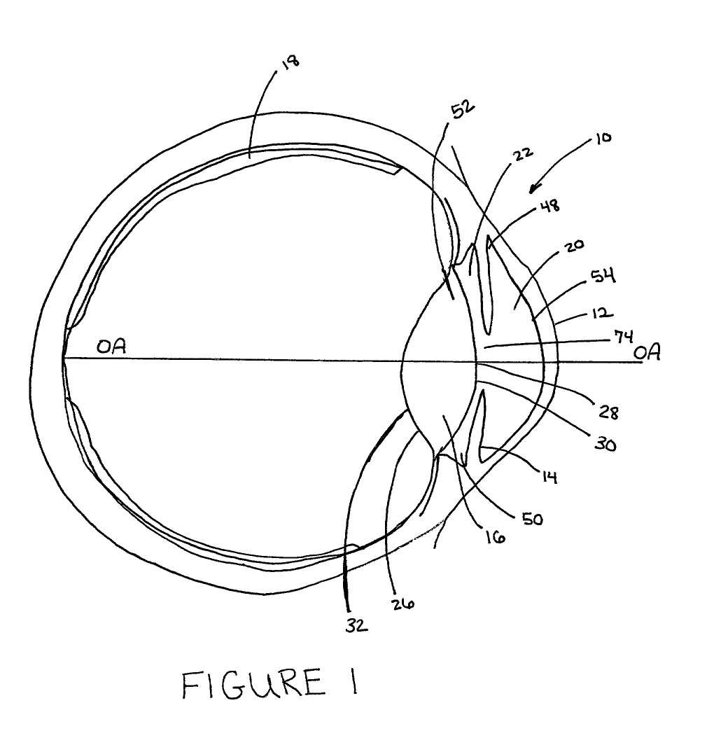

FIG. 1 illustrates a simplified diagram of an eye 10 showing landmark structures relevant to the implantation of an intraocular lens (IOL) of the present invention. Eye 10 includes an optically clear cornea 12 and an iris 14. A natural crystalline lens 16 and a retina 18 are located behind the iris 14 of eye 10. Eye 10 also includes anterior chamber 20 located in front of iris 14 and a posterior chamber 22 located between iris 14 and natural lens 16. An IOL 24, such as that of the present invention, is preferably implanted in anterior chamber 20 to correct refractive errors while healthy natural lens 16 remains in place (phakic application). IOLs of the present invention may also be implanted in posterior chamber 22 or lens capsule 26 for use in aphakic eyes. When used in aphakic eyes, IOLs serve as replacements for surgically removed diseased natural lenses 16, such as for example following cataract surgeries. Eye 10 also includes an optical axis OA-OA that is an imaginary line that passes through the optical center 28 of anterior surface 30 and posterior surface 32 of lens 16. Optical axis OA-OA in the human eye 10 is generally perpendicular to a portion of cornea 12, natural lens 16 and retina 18.

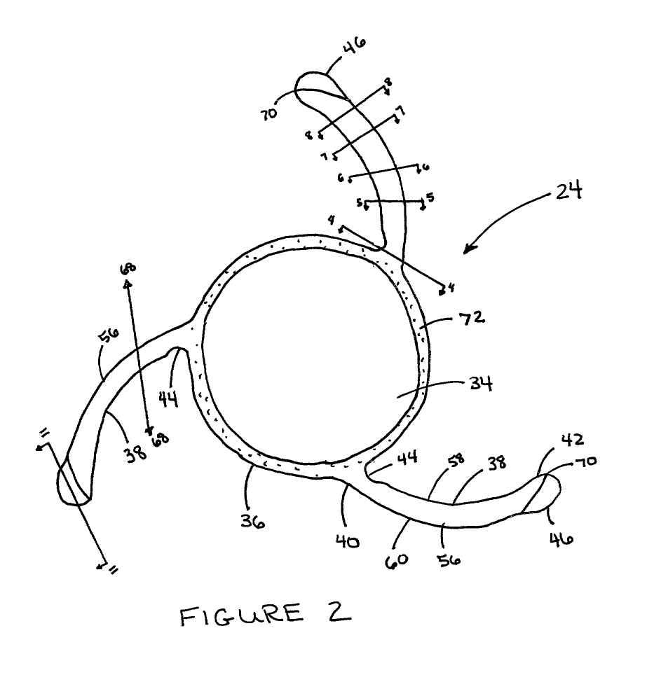

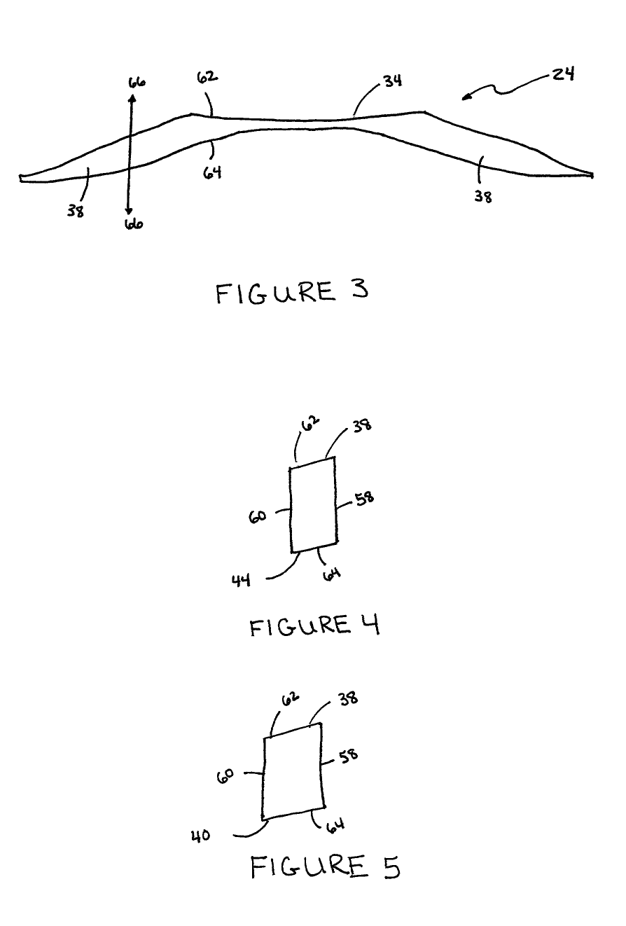

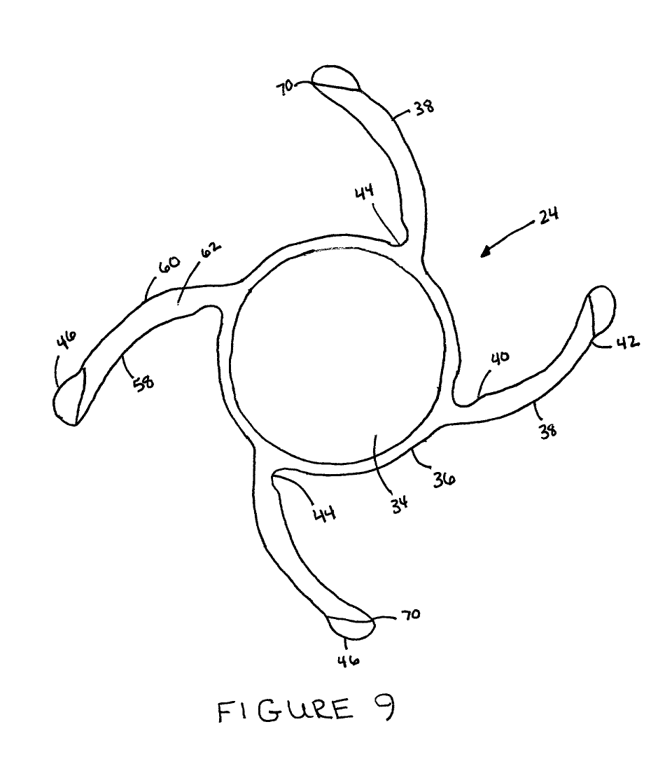

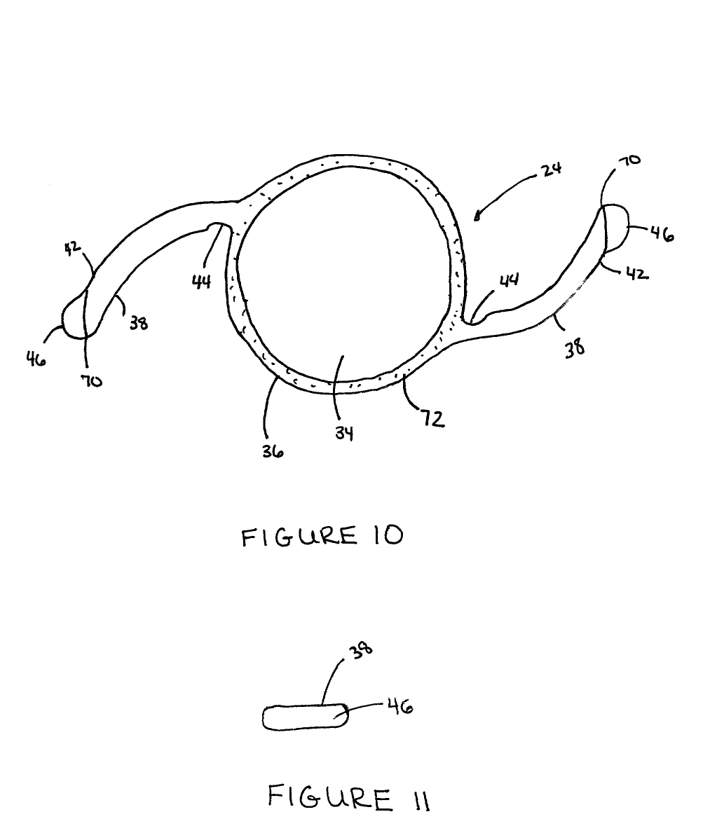

IOLs of the present invention, illustrated in FIGS. 2, 9 and 10, identified generally by reference numeral 24, are designed for implantation preferably in anterior chamber 20 of a patient's eye 10. However as mentioned above, IOL 24 may likewise be implanted in posterior chamber 22 or in the case of an aphakic eye, in lens capsule 26. IOL 24 has an optic portion 34 with an outer peripheral edge 36. Preferably integrally formed on peripheral edge 36 of optic portion 34 are two or more but preferably two, three or four separate haptic elements 38. Each haptic element 38 has like form to achieve a “propeller-like” appearance for ease in turning and centering IOL 24 upon implantation within an eye and to achieve the desired inward flexation into closer proximity with optic portion 34 and optic portion 34 rotation as described in greater detail below.

Each haptic element 38 is manufactured to have an inner portion 40 and an outer portion 42. Inner portion 40 of each haptic element 38 includes a gusset portion 44 preferably integrally formed with and permanently connected to outer peripheral edge 36 of optic portion 34. Alternatively however, gusset portion 44 of each haptic element 38 may be permanently attached to optic portion 34 by staking, chemical polymerization or other methods known to those skilled in the art. Each haptic element 38 also includes on outer portion 42 adjacent to center tip 70, a softened contact button 46 designed to preferably engage inner surfaces 48 in anterior chamber 20. However, contact buttons 46 are also suitable to engage inner surfaces 50 in posterior chamber 22 or inner surfaces 52 in lens capsule 26 of an aphakic eye 10.

When IOL 24 is implanted in a patient's phakic or aphakic eye 10 and held in place through compressive forces exerted by inner surfaces 48, 50 or 52 on contact buttons 46, haptic elements 38 flex. Upon inward flexation of haptic elements 38 into closer proximity to optic portion 34, optic portion 34 rotates. Due to this rotational release of energy through optic portion 34, contact buttons 46 remain in constant, non-sliding contact with inner surfaces 48, 50 or 52 of eye 10. Sliding contact and/or intermittent contact of contact buttons 46 with inner surfaces 48, 50 or 52 is thus avoided in the subject IOL 24 to minimize or eliminate tissue damage within eye 10.

To achieve the desired flexation characteristics from haptic elements 38, the same must flex in a plane generally parallel to that of optic portion 34 and generally perpendicular to that of optical axis OA-OA. The flexation characteristics of haptic elements 38 with optic portion 34 rotation minimize axial displacement of optic portion 34 in a direction along optical axis OA-OA of eye 10. Compressive forces of differing magnitudes within the range of approximately 0.2 to 0.8 mN exerted against contact buttons 46 of haptic elements 38 to effect approximately an overall 1.0 mm in diameter compression of IOL 24, such as that caused by differing eye sizes, results in less than approximately 1.0 mm, but more preferably less than approximately 0.5 mm and most preferably less than approximately 0.3 mm axial displacement of optic portion 34 along optical axis OA-OA in eye 10. Under like compressive forces, IOLs known in the art result in approximately 2.0 mm axial displacement of the optic portion along the optical axis, which may damage delicate eye tissues. The unique low profile design of IOL 24 achieves minimized axial displacement of optic portion 34 to protect the corneal endothelium 54 of eye 10 from harmful contact and damage when compressive forces are applied to eye 10.

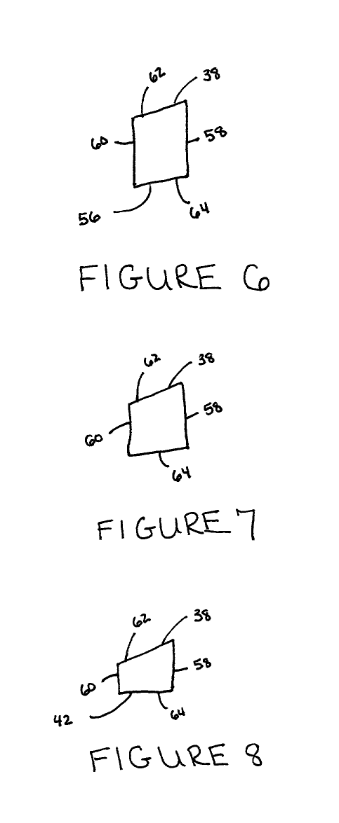

The flexation characteristics of haptic elements 38 with rotation of optic portion 34 as described above is achieved through a unique low profile design. IOL 24 has haptic elements 38 formed with elongated arcuate central portions 56 that extend between inner portions 40 and center tips 70 of outer portions 42. As best illustrated in FIGS. 4 through 9, from gusset portion 44 throughout the length of elongated arcuate central portion 56, the cross-section of haptic element 38 is trapezoidal in shape with fluidly varying dimensions to achieve the desired flexation characteristics while maintaining a relatively low profile. By designing haptic elements with a trapezoidal shape, IOL 24 achieves a thinner, lower profile at outer edge surface 60 as dictated by the limited space of anterior chamber 20, than would otherwise be achievable. Haptic element 38 varies in dimension from gusset portion 44 throughout the length of central portion 56 in both plane 66-66 generally parallel to optical axis OA-OA, and plane 68-68 generally perpendicular to optical axis OA-OA. Moving from gusset portion 44 toward outer portion 42, haptic element 38 varies in thickness in plane 66-66. Inner edge surface 58 at gusset 44 is approximately 0.800 to 0.804 mm in thickness, or approximately 1 to 2 percent smaller in dimension than that of parallel outer edge surface 60 which is approximately 0.810 to 0.814 mm in thickness, as illustrated in FIG. 4. As illustrated in FIG. 5, at inner portion 40, inner edge surface 58 is approximately 0.803 to 0.807 mm in thickness, or approximately 0.25 to 1 percent thicker than the same at gusset portion 44. At inner portion 40, outer edge surface 60 is approximately 0.776 to 0.780 mm in thickness or approximately 4 to 5 percent thinner than the same at gusset portion 44. At inner portion 40, inner edge surface 58 is approximately 4 to 5 percent larger in dimension than that of parallel outer edge surface 60. Illustrated in FIG. 6, at approximately the center of haptic element 38, inner edge surface 58 is approximately 0.737 to 0.741 mm in thickness, which is approximately 8 to 9 percent thinner than the same at inner portion 40. At approximately the center of haptic element 38, outer edge surface 60 is approximately 0.668 to 0.672 mm in thickness, which is approximately 13 to 14 percent thinner than the same at inner portion 40. At approximately the center of haptic element 38, inner edge surface 58 is approximately 9 to 10 percent larger in dimension than that of parallel outer edge surface 60. Illustrated in FIG. 7, at outer portion 42, inner edge surface 58 is approximately 0.625 to 0.629 mm in thickness, which is approximately 15 to 16 percent thinner than the same at approximately the center of haptic element 38. At outer portion 42, outer edge surface 60 is approximately 0.503 to 0.507 mm in thickness, which is approximately 24 to 25 percent thinner than the same at approximately the center of haptic element 38. At outer portion 42, inner edge surface 58 is approximately 19 to 20 percent larger in dimension than that of parallel outer edge surface 60. Illustrated in FIG. 8, at outer portion 42 adjacent contact button 46, inner edge surface 58 is approximately 0.475 to 0.479 mm in thickness, which is approximately 23 to 24 percent thinner than the same at outer portion 42. At outer portion 42 adjacent contact button 46, outer edge surface 60 is approximately 0.289 to 0.293 mm in thickness, which is approximately 42 to 43 percent thinner than the same at outer portion 42. At outer portion 42 adjacent contact button 46, inner edge surface 58 is approximately 38 to 39 percent larger in dimension than that of parallel outer edge surface 60.

Moving from gusset portion 44 toward outer portion 42, haptic element 38 varies in width in plane 68-68. At gusset 44, anterior surface 62 and posterior surface 64 are of an equal width of approximately 0.459 to 0.463 mm, which is approximately 43 to 44 percent smaller in width in plane 68-68 than the thickness in plane 66 - 66 of outer edge surface 60 as illustrated in FIG. 4. As illustrated in FIG. 5, at inner portion 40, anterior surface 62 and posterior surface 64 are of an equal width of approximately 0.582 to 0.584 mm, which is approximately 21 to 22 percent wider than the same at gusset 44. At inner portion 40, anterior surface 62 and posterior surface 64 are approximately 27 to 28 smaller in width in plane 68-68 than inner edge surface 58 is in thickness in plane 66-66. Illustrated in FIG. 6, at approximately the center of haptic element 38, anterior surface 62 and posterior surface 64 are of an equal width of approximately 0.547 to 0.551 mm, which is approximately 5 to 6 percent narrower than the same at inner portion 40. At approximately the center of haptic element 38, anterior surface 62 and posterior surface 64 are approximately 25 to 26 percent smaller in dimension than that of inner edge surface 58. Illustrated in FIG. 7, at outer portion 42 anterior surface 62 and posterior surface 64 are of an equal width of approximately 0.532 to 0.536 mm, which is approximately 2 to 3 percent narrower than the same at approximately the center of haptic element 38. At outer portion 42, anterior surface 62 and posterior surface 64 are approximately 14 to 15 percent smaller in dimension than that of inner edge surface 58. Illustrated in FIG. 8, at outer portion 42 adjacent contact button 46, anterior surface 62 and posterior surface 64 are of an equal width of approximately 0.531 to 0.535 mm, which is approximately the same dimension as the same at outer portion 42. At outer portion 42 adjacent contact button 46, anterior surface 62 and posterior surface 64 are approximately 10 to 11 percent larger in dimension than that of inner edge surface 58.

In general, in moving from gusset 44 to outer portion 42, haptic elements 38 first increase and then decrease in thickness in plane 66-66 to form a convex-convex cross sectional shape. Such haptic element 38 dimensions allow the thickness in plane 66-66 to be maximized. By maximizing thickness in plane 66-66, the aspect ratio defined as width divided by thickness, is minimized. As the aspect ratio decreases, axial displacement of optic portion 34 so too decreases.

Contact buttons 46 are relatively small rounded elongated protuberances formed on outer edge surface 60 at outer portion 42 as best illustrated in FIGS. 2 and 11. Contact buttons 46 have a width in plane 68-68 at outer edge surface 60 of approximately 0.75 to 1.25 mm. The thickness in plane 66-66 of contact buttons 46 continually decreases moving in a direction away from outer edge surface 60. This decrease in thickness allows contact buttons 46 to fit perfectly within the angle or inner surface 48 of anterior chamber 20. Contact buttons 46 are so formed as relatively small rounded elongated protuberances to maintain constant, non-sliding contact with the delicate tissues of inner surface 48.

The subject IOL 24 is preferably produced having an optic portion 34 approximately 4.5 to 9.0 mm, but preferably approximately 5.0 to 6.0 mm and most preferably 5.5 mm in diameter and approximately 0.5 mm to 1.0 mm, but preferably approximately 0.6 to 0.8 mm and most preferably 0.7 mm in thickness at peripheral edge 36. Haptic elements 38 extend in an overall arcuate configuration and increase or decrease in overall length depending upon the diameter of optic portion 34. As the diameter of optic portion 34 increases, the overall length of haptic elements 38 decrease. Likewise, as the diameter of optic portion 34 decreases, the overall length of haptic elements 38 increase. In general, haptic elements 38 are formed to be approximately 2.6 to 6.0 mm, but preferably approximately 3.4 to 5.5 mm and most preferably approximately 4.8 mm in length measuring from the center of inner portion 40 to the center tip 70 of outer portion 42. Haptic elements 38 have an arcuate configuration as illustrated in FIGS. 2, 9 and 10 to allow radial deflection under compressive forces while contact buttons 46 maintain constant, non-sliding contact within eye 10. For purposes of the present invention, the arcuate shape of haptic element 38, i.e., the beam curve shape, relative to the width to thickness ratio, i.e., the aspect ratio, of haptic element 38 as described herein is critical to achieve suitable function. Central portion 56 of haptic element 38 is approximately 0.6 to 5.0 mm, but preferably approximately 1.4 to 4.5 mm and most preferably approximately 4.0 mm in length. Inner portion 40 comprising gusset portion 44 is approximately 0.25 to 1.5 mm, but preferably approximately 0.50 to 0.125 mm and most preferably approximately 0.80 mm in length.

As provided through the dimensions of IOL 24, haptic elements 38 gradually change from being relatively thin in plane 66-66 at outer portion 42 to being relatively thick at inner portion 40. Central portions 56 exhibit a thicker dimension in plane 66-66 along inner edge surface 58 than along outer edge surface 60. Central portion 56 likewise exhibits a thicker dimension in plane 66-66 than that of the width in plane 68-68. Haptic elements 38 of the subject design tend to flex at gusset portions 44 due to a reduced width at gusset portions 44 in plane 68-68. Upon flexation of haptic elements 38 at gusset portions 44, inner edge surface 58 moves into closer proximity with outer peripheral edge 36. Accordingly, when a compression force is exerted against contact buttons 46, haptic elements 38 flex causing rotational movement of optic portion 34. Any radial displacement energy applied to haptic elements 38 is converted into rotational energy released through rotation of optic portion 34. By releasing any radial displacement energy through rotation of optic portion 34, axial displacement of optic portion 34 along optical axis OA-OA is minimized. When IOL 24 is used as a refractive lens, a stable, reliable refractive correction is thereby provided.

Suitable materials for the production of the subject IOL 24 include but are not limited to foldable or compressible materials, such as silicone polymers, hydrocarbon and fluorocarbon polymers, hydrogels, soft acrylic polymers, polyesters, polyamides, polyurethane, silicone polymers with hydrophilic monomer units, fluorine-containing polysiloxane elastomers and combinations thereof. The preferred material for the production of IOL 24 of the present invention is a hydrophilic or hydrophobic acrylic material such as those known to those skilled in the art. Poly(HEMA-co-HOHEXMA) is a preferred hydrophilic acrylic material useful for the manufacture of IOL 24 due to its equilibrium water content of approximately 18 percent by weight and high refractive index of approximately 1.474, which is greater than that of the aqueous humor of the eye, i.e., 1.33. High refractive index is a desirable feature in the production of IOLs to impart high optical power with a minimum of optic thickness. By using a material with a high refractive index, visual acuity deficiencies may be corrected using a thinner IOL. A thin or low profile IOL, such as that of IOL 24, is particularly desirable in phakic applications to minimize potentially harmful contact between the IOL and iris 14 and corneal endothelium 54. Poly(HEMA-co-HOHEXMA) is also a desirable material in the production of IOL 24 due to its mechanical strength, which is suitable to withstand considerable physical manipulation. Poly(HEMA-co-HOHEXMA) also has desirable memory properties suitable for IOL use. IOLs manufactured from a material possessing good memory properties such as those of poly(HEMA-co-HOHEXMA) unfold in a controlled manner in an eye, rather than explosively, to its predetermined shape. Explosive unfolding of IOLs is undesirable due to potential damage to delicate tissues within the eye. Poly(HEMA-co-HOHEXMA) also has dimensional stability in the eye.

IOL 24 may likewise be manufactured using a variety of materials having various physical characteristics. For example, IOL 24 may be manufactured to have an optic portion 34 of a high refractive index hydrophilic acrylic material, haptic elements 38 of a material more rigid than that of optic portion 34 and contact buttons of the same material as that of optic portion 34 or a differing material of a lower refractive index and a greater glass transition temperature.

Although the teachings of the present invention are preferably applied to soft or foldable IOLs formed of a foldable or compressible material, the same may also be applied to harder, less flexible lenses formed of a relatively rigid material such as polymethylmethacrylate (PMMA) having flexible haptics formed either of the same or a different material.

Optic portion 34 of IOL 24 can be a positive powered lens from 0 to approximately +40 diopters or a negative powered lens from 0 to approximately −30 diopters. Optic portion 34 may be biconvex, plano-convex, plano-concave, biconcave or concave-convex (meniscus), depending upon the power required to achieve the appropriate central and peripheral thickness for efficient handling.

Optic portion 34 of the subject IOL 24 may optionally be formed with a glare reduction zone 72 of approximately 0.25 to 0.75 mm but more preferably approximately 0.3 to 0.6 mm and most preferably 0.5 mm in width adjacent outer peripheral edge 36 for reducing glare when outer peripheral edge 36 of IOL 24 is struck by light entering eye 10 during high light or at other times when pupil 74 is dilated. Glare reduction zone 72 is typically fabricated of the same material as optic portion 34, but may be opaque, colored or patterned in a conventional manner to block or diffuse light in plane with optical axis OA-OA.

Subject IOL 24 is preferably manufactured by first producing single material or multiple material discs from one or more materials of choice as described in U.S. Pat. Nos. 5,217,491 and 5,326,506 each incorporated herein in its entirety by reference. IOL 24 may then be machined from the material discs in a conventional manner. Once machined, IOL 24 may be polished, cleaned, sterilized and packaged by a conventional method known to those skilled in the art.

Subject IOL 24 is used in eye 10 by creating an incision in cornea 12, inserting IOL 24 in either anterior chamber 20 or posterior chamber 22 and closing the incision in accordance with methods known to those skilled in the art. Alternatively, IOL 24 may be used in eye 10 by creating an incision in cornea 12 and lens capsule 26, removing natural lens 16, inserting IOL 24 in lens capsule 26 and closing the incision in accordance with methods known to those skilled in the art.

IOL 24 of the present invention provides for a refractive lens suitable for use in lens capsule 26 or posterior chamber 22, but most preferably due to its relatively low profile for use in anterior chamber 20 of eye 10. IOL 24 has haptic elements 38 with flexibility characteristics that minimize axial displacement along optical axis OA-OA of eye 10 thereby preventing decentration of IOL 24, distortion of vision and damage to corneal endothelium 54. IOL 24, having the rotational flexibility characteristics described herein is also advantageous because one or a few lens sizes suitably fit eyes 10 of most sizes. By providing a “universal” lens such as that of the present invention, clinical risks to patients due to improperly sized lenses are minimized. Such clinical risks minimized include pupil ovalization, corneal endothelium damage and poor fixation. Likewise, manufacturers' need to produce IOLs of many sizes to fit eyes of many sizes is eliminated, thus reducing production and inventory costs associated therewith. Ophthalmologists also benefit from subject IOL 24 in that time is saved by eliminating the need to determine each patient's eye size and costs associated with maintaining large inventories of varying sized lenses.

While there is shown and described herein certain specific embodiments of the present invention, it will be manifest to those skilled in the art that various modifications may be made without departing from the spirit and scope of the underlying inventive concept and that the same is not limited to the particular forms herein shown and described except insofar as indicated by the scope of the appended claims.

We claim:

1. An intraocular lens to be implanted within an eye generally perpendicular to the eye's optical axis comprising:
an outer peripheral edge defining an optic portion, and two or more haptic elements of like fluidly varying trapezoidal form permanently connected through a gusset portion to said outer peripheral edge,
whereby a compressive force sufficient to effect a 1.0 mm in diameter compression of said lens results in less than approximately 1.0 mm of axial displacement of said optic portion along the eye's optical axis.

2. An intraocular lens to be implanted within an eye generally perpendicular to the eye's optical axis comprising:
an outer peripheral edge defining an optic portion, and two or more haptic elements of like fluidly varying trapezoidal form permanently connected through a gusset portion to said outer peripheral edge,
whereby a compressive force sufficient to effect a 1.0 mm in diameter compression of said lens results in less than approximately 0.5 mm of axial displacement of said optic portion along the eye's optical axis.

3. An intraocular lens to be implanted within an eye generally perpendicular to the eye's optical axis comprising:
an outer peripheral edge defining an optic portion, and two or more haptic elements of like fluidly varying trapezoidal form permanently connected through a gusset portion to said outer peripheral edge,
whereby a compressive force sufficient to effect a 1.0 mm in diameter compression of said lens results in less than approximately 0.3 mm of axial displacement of said optic portion along the eye's optical axis.

4. The intraocular lens of claim 1, 2 or 3 wherein the haptic elements and the optic portion are both formed of a foldable or compressible material.

5. The intraocular lens of claim 1, 2 or 3 wherein said lens is formed from a material selected from the group consisting of silicone polymers, hydrocarbon and fluorocarbon polymers, hydrogels, soft acrylic polymers, polyester, polyamides, polyurethane, silicone polymers with hydrophilic monomer units, fluorine-containing polysiloxane elastomers and combinations thereof.

6. The intraocular lens of claim 1, 2 or 3 wherein said lens is formed from a hydrophilic acrylic material.

7. The intraocular lens of claim 1, 2 or 3 wherein said lens is formed from a hydrophilic acrylic material which is 18 percent by weight water.

8. The intraocular lens of claim 1, 2 or 3 wherein said lens is formed from poly(HEMA-co-HOHEXMA).

9. The intraocular lens of claim 1, 2 or 3 wherein said lens is formed from one or more materials with at least one material having a refractive index above 1.33.

10. The intraocular lens of claim 1, 2 or 3 wherein said lens is formed from one or more materials with at least one material being an acrylic material.

11. The intraocular lens of claim 1, 2 or 3 wherein said lens is formed from one or more materials with at least one material being a silicone material.

12. The intraocular lens of claim 1, 2 or 3 wherein said haptic elements are dimensioned to be smaller in a plane generally perpendicular to the eye's optical axis than in a plane generally parallel to the eye's optical axis at an inner portion thereof, and dimensioned to be the same or greater in a plane generally perpendicular to the eye's optical axis than in a plane generally parallel to the eye's optical axis at an outer portion thereof.

13. The intraocular lens of claim 1, 2 or 3 wherein a glare reduction zone is formed adjacent to the outer peripheral edge of the optic portion.

14. A method of manufacturing the intraocular lens of claim 1, 2 or 3 comprising:
forming a disk of one or more suitable materials, and
machining said lens from said disk.

15. A method of using the intraocular lens of claim 1, 2 or 3 comprising:
creating an incision in a cornea of an eye, and
inserting said intraocular lens into an anterior chamber of said eye.

16. A method of using the intraocular lens of claim 1, 2 or 3 comprising:
creating an incision in a cornea of an eye, and
inserting said intraocular lens into a posterior chamber of said eye.

17. A method of using the intraocular lens of claim 1,2 or 3 comprising:
creating an incision in a cornea and lens capsule of an eye,
removing a natural lens of said eye, and
inserting said intraocular lens into said lens capsule of said eye.
